(19) United States
(12) Patent Application Publication
(10) Pub. No.: US 2017/0233271 A1
(43) Pub. Date: Aug. 17, 2017

(54) PROCESS FOR ENHANCING POLYHYDROXYALKANOATE ACCUMULATION IN ACTIVATED SLUDGE BIOMASS

(71) Applicants: Alan Gideon WERKER, Lomma (SE); Simon Olof Harald BENGTSSON, Dalby (SE); John Markus HJORT, Kungsbacka (SE); Fernando MORGAN-SAGASTUME, Malmö (SE); Mauro MAJONE, Roma (IT); Francesco VALENTINO, Roma (IT); VEOLIA WATER SOLUTIONS & TECHNOLOGIES SUPPORT, SAINT MAURICE (FR); UNIVERSITY OF ROME, Roma (IT)

(72) Inventors: Alan Gideon Werker, Lomma (SE); Simon Olof Harald Bengtsson, Dalby (SE); John Markus Hjort, Kungsbacka (SE); Fernando Morgan-Sagastume, Malmö (FR); Mauro Majone, Roma (IT); Francesco Valentino, Roma (IT)

(73) Assignee: VEOLIA WATER SOLUTIONS & TECHNOLOGIES SUPPORT, SAINT MAURICE (FR)

(21) Appl. No.: 15/502,052

(22) PCT Filed: Aug. 6, 2015
(86) PCT No.: PCT/IB2015/055993

§ 371 Date: Feb. 6, 2017

Related U.S. Application Data
(60) Provisional application No. 62/034,233, filed on Aug. 7, 2014.

(51) Int. Cl. C02F 3/12; C12P 7/62

(52) U.S. Cl. CPC C02F 3/1205; C02F 3/1263; C12P 7/625; C02F 2209/06; C02F 2305/06; C02F 2209/08; C02F 2203/004

(57) ABSTRACT

A process for producing PHA comprises obtaining biomass produced in the course of biologically treating a first wastewater source containing RBCOD. The biomass is to be exploited with a second wastewater source having a different RBCOD content from the first wastewater source in order to accumulate and thereby produce PHA. Before subjecting the biomass to a PHA accumulation process, the biomass PHA accumulation potential is enhanced via an acclimation process with the second wastewater source. During acclimation, the biomass is subjected to repeated feast-famine periods. During each feast period, the biomass is exposed to a fraction of the second wastewater source. The RBCOD uptake and/or biomass respiration rate is directly or indirectly measured during each feast period. The famine period is maintained for a period of time that is at least two times greater than the length of time of the proceeding feast period. After at least two feast-famine acclimation periods or after one or more measured parameters reveal an increased RBCOD relative uptake or respiration rate of the biomass during a subsequent feast period, the biomass is subjected to a PHA accumulation process using the second wastewater source.

### TECHNICAL FIELD

The methods disclosed herein relate to biologically treating process water and/or wastewater streams and accumulating PHA.

### BACKGROUND

In biological wastewater treatment, chemical oxygen demand (COD) is removed by growing microbial biomass and the surplus biomass produced becomes a process waste by-product. This waste biomass requires management and disposal, and that represents major operating costs. An alternative method to valorize this surplus biomass is by using it for producing biodegradable polymers. This alternate route has attracted much interest since this still allows for wastewater treatment while yielding a raw material, rather than a waste. This biomass raw material can be used to produce value-added biopolymers and the left over biomass after the biopolymer recovery can also be further exploited as a resource.

The biopolymers produced by biomass treating wastewater are polyhydroxyalkanoates (PHAs), which are a group of polyesters that may become accumulated as intracellular carbon and energy reservoirs by many species of naturally occurring bacteria. PHAs can be recovered from biomass and converted into biodegradable plastics of commercial value with a broad range of practical applications. See, for example, US 2010/0200498, WO 2011/070544A2, WO 2011/073744A1, WO 2012/022998A1, WO 2012/023114A1.

So-called mixed-culture PHA production may be seen as an element or consequence of wastewater treatment involving three main process elements PE1 to PE3: (PE1) the sourcing of organic rich streams dominated by readily biodegradable chemical oxygen demand (RBCOD) that may involve the fermentation of wastewaters and sludge feedstocks; (PE2) the production of a biomass, with some (to greater or lesser degree) PHA accumulation potential (PAP), during a wastewater treatment; and (PE3) the production of PHA using the output of PE1 (RBCOD rich feedstock) and PE2 (surplus biomass) as input raw materials.

The PE1 output is an effluent wastewater that may be fed to a PE2 in order to produce a biomass with PAP. However, it is advantageous to produce a biomass with PAP in a PE2 by treating and exploiting, for example, a municipal wastewater wherein the municipal wastewater is sufficient for producing a biomass with PAP but it is insufficient as an input to a PE3. In cases where the PE1 output is a RBCOD rich water that is different from the PE2 input water treated and used to produce a biomass, there may be need to acclimate the biomass to the feedstock used in the PE3.

While a biomass exhibiting significant PAP can generally be harvested from the wastewater treatment process (PE2), there remains a need to increase the ability of the biomass to accumulate a maximum amount of PHA with high average molecular mass. The present invention concerns methods and process of biomass acclimation after a PE2 and in advance of a PE3 wherein the biomass accumulation potential in the PE3 is enhanced.

### SUMMARY

One embodiment of the present invention addresses a process for producing PHA and comprises obtaining biomass produced in the course of biologically treating a first wastewater source containing RBCOD. The biomass is mixed with a second wastewater source having a different RBCOD content from the RBCOD content of the first wastewater source. Before subjecting the biomass to a PHA accumulation process, the biomass PHA accumulation potential is enhanced via an acclimation process with the second wastewater source. The biomass is subjected to repeated feast-famine periods. During each feast period, the biomass is exposed to at least a fraction of the second wastewater source. The RBCOD uptake and/or biomass respiration rate is directly or indirectly measured during the feast period. After the biomass has removed 90% or more of the RBCOD in the added fraction of the second wastewater, the biomass is subjected to a period of famine. The famine period is maintained for a period of time that is at least two times greater than the length of time of the proceeding feast period. After at least two feast-famine periods in the acclimation process or after one or more measured parameters reveal an increased RBCOD uptake rate of the biomass during a feast period, or an increased biomass respiration rate during a feast period, the biomass is subjected to a PHA accumulation process. The PHA accumulation process utilizes the second wastewater source to produce and accumulate PHA in the biomass derived from treating the first wastewater source.

Another embodiment discloses a method of treating wastewater and producing PHA. A first wastewater stream having RBCOD content is biologically treated and a biomass is produced. A second wastewater stream containing RBCOD content that is different in RBCOD content to the first wastewater stream is utilized to acclimate the biomass. In acclimation, the biomass is stimulated into a period of feast respiration by subjecting the biomass to a fraction of wastewater from the second wastewater stream and treating this faction of added wastewater. During the period of feast respiration, the RBCOD uptake rate or biomass respiration rate are measured directly or indirectly. After the biomass removes at least 90% of the added RBCOD from the wastewater fraction, the biomass is subjected to a period of famine. The period of famine is at least twice as long as the period of feast. The biomass is repeatedly subjected to periods of feast respiration and periods of famine for at least two feast-famine cycles and until either the substrate uptake rate is at least 10% higher than the substrate uptake rate during the first period of feast respiration, or the respiration rate is at least 10% higher than the respiration rate during the first period of feast respiration. Thereafter, more of the second wastewater stream is biologically treated while accumulating PHA in a batch process. PHA accumulation is accomplished by subjecting the biomass to a prolonged feast respiration with the second wastewater stream.

The acclimation process is not a process of biomass production, nor is it a process of PHA accumulation. The biomass during acclimation is to a large extent as possible retained in the acclimation process and the amount of biomass in the process does not increase by more than a small fraction of the amount biomass that is introduced into the process. The retention time of the biomass in the acclimation process is generally less than the solids retention times used to produce the biomass in a PE2. Further, the content of PHA in the biomass does not increase by more than a few percent during the acclimation process. The purpose of the acclimation process is to acclimate the biomass to the second wastewater in such a way as to elicit a stronger PHA storage response, with respect to rate of PHA accumulation and amount of PHA accumulation, when the biomass transitions from the process of acclimation to a process of accumulation.

### BRIEF DESCRIPTION OF THE DRAWINGS

FIG. 1 depicts a graph showing the production of PHA and active biomass (Xa), consumption of acetate, and biomass PHA content during the accumulation runs without nutrient addition and acclimation (A), with nutrient addition and without acclimation (B), and with nutrient addition and acclimation (C). For (C), the negative time indicates the duration of the step of acclimation previous to the accumulation.

FIG. 2 depicts a graph showing the production of PHA and active biomass (Xa), consumption of an acetate-propionate mixture, and biomass PHA content during the accumulation runs without acclimation (A) and with a step of acclimation (B). For (B), the negative time indicates the duration of the acclimation step previous to the accumulation.

FIG. 3 depicts a graph showing feast duration and specific rates of substrate (RBCOD) consumption and PHA storage during the feast (A), and PHA and active biomass (Xa) produced during 16 feast-famine cycles with an acetate and propionate mixture with nutrients in excess as substrates.

FIG. 4 depicts a schematic example of process elements of biomass production (PE2) and PHA-production (PE3) with benefit of improved biomass PHA accumulation potential due to an intervening step of feast-famine acclimation.

FIG. 5 depicts a graph showing the production of PHA and active biomass (Xa), consumption of fermented-sludge liquor substrate, and biomass PHA content during the accumulation runs with and without a step of acclimation (PA).

FIG. 6 depicts a graph showing the development of specific substrate uptake rates and feast duration during the acclimation step. The ratios of the substrate uptake rates (qᵢ/q₁) and feast durations (pᵢ/p₁) are presented where i corresponds to the cycle number. Each data point corresponds to the time of feast start, and the continuous line to fitted data to a quadratic equation with minimized sum of squared errors.

FIG. 7 depicts a graph showing the characterization of feast response of biomass with and without an acclimation step based on dissolved oxygen (DO) (A), pH and COD (B), and N species (C) profiles measured in batch. The initial DO values for the batch feast tests with and without acclimation were 7.3 and 8.1, respectively.

FIG. 8 depicts a graph showing the development of specific substrate uptake rates and feast duration during the acclimation step. The ratios of the substrate uptake rates (qᵢ/q₁) and feast durations (pᵢ/p₁) are presented where i corresponds to the cycle number. Each data point corresponds to the time of feast start, and the continuous line to fitted data to an exponential equation with minimized sum of squared errors.

FIG. 9 depicts a graph showing the trend of acclimation step biomass specific substrate uptake rate with repeated cycles of feast stimulation and famine with a feast-famine time ratio of 0.125.

FIG. 10 depicts a graph showing the trend of acclimation step feast time with repeated cycles of feast stimulation and famine with a feast-famine time ratio of 0.125.

FIG. 11 depicts a graph showing the correlation of acclimation step biomass feast specific substrate uptake rate versus specific oxygen uptake rate with repeated cycles of feast stimulation and famine with a feast-famine time ratio of 0.125.

### DETAILED DESCRIPTION

This application relates to methods and processes for conditioning a biomass after the biomass has been produced but before the biomass is used to produce PHA, in order to improve the PHA production performance of the biomass in the PHA production process. The biomass is conditioned in an acclimation process and after the biomass has undergone acclimation, the biomass is subjected to a PHA production process. Both the acclimation process and the PHA production process is carried out in the course of treating wastewater. The term “wastewater”, as used herein, includes all types of wastewater and process waters. Process waters are typically a by-product of an industrial process and may, in part, be recycled back within the industrial process after treatment. An example is a paper mill where the water used for pulping and papermaking may be used and re-used in the process and as part of the process water management there is a step to remove some contamination from at least some of the process water so that it may be reused again. Wastewater is effluent water produced as waste from municipal or industrial activity. It is an effluent that is to be used or discharged to the receiving environment after some form of treatment. As both process waters and wastewaters may be applicable to the methods and processes described herein, the expression “wastewater” is used without limitation to refer to any influent water to the methods described herein, in general, and wherein treatment refers to water quality improvement of at least the reduction of the influent water organic content.

One embodiment entails a first step of obtaining biomass produced from treating a first stream, namely a wastewater. Therefore, this first stream produces the biomass that is to be used as an active raw material for a PHA production process. The other raw material for the PHA production process is a source of RBCOD that is supplied, at least in part, by a second stream. The next step is to expose this biomass to an acclimation process wherein the biomass is subjected to this second stream that is distinct in water quality from the first stream. This is referred to as an acclimation process step because the biomass is disposed to become acclimatized to the second stream before being disposed to a process of PHA accumulation wherein the biomass performance in PHA accumulation becomes enhanced by the methods of acclimation described herein. Generally the key difference between the first and second streams is with respect to the soluble organic composition, and more specifically the RBCOD content of the two streams. During the acclimation process, the biomass is repeatedly stimulated into a feast respiration and each feast respiration is followed by a famine time that is more than twice as long as the time to which the biomass is in feast for each respective stimulation. Cycles of feast stimulation followed by famine are repeated until at least one criteria of increased rate of response is achieved by the biomass or until a maximum number of feast stimulations is applied. Generally, at least two, but preferably more, feast stimulations are to be applied. This stimulation is to acclimate the biomass in preparation to a subsequent PHA accumulation process to be conducted with a feedstock that includes the second stream. The time taken for the biomass acclimation is much shorter than the SRT of the process used to produce the biomass on the first stream. In clear distinction to processes of feast-famine enrichment, feast-famine in the case of said acclimation step is not with a purpose to selectively produce biomass rich with PHA accumulating potential; feast-famine in the acclimation step is with the objective to elicit a stronger PHA accumulating potential in the extant biomass. The acclimation step is a process to condition the extant biomass. Further, most but ideally all of the biomass is retained during the process of acclimation. Retention of the biomass makes distinction of goals with respect to processes of feast-famine selection that are directed to enriching a bacterial ecosystem with the PHA storing phenotype, versus disclosed methods of feast-famine acclimation directed to conditioning of an extant biomass physiological state. The biomass is retained furthermore as much as possible because it is a resource raw material for producing the PHA as a value added product. Then, including the second stream in a PHA production process, we accumulate PHA in the acclimated biomass. It has been found that by means of the intermediary conditioning step of acclimation we are able to prepare the biomass in order to significantly improve productivity in the PHA accumulation process.

We have discovered that the full expression of a biomass PHA accumulation potential (PAP) may not only rely on the microbial composition of the biomass selected by the wastewater treatment. This extent of PHA, which the biomass may accumulate in the PE3 PHA production process, may be further related to the condition or physiological state of the biomass at the beginning of this PE3 biopolymer production step. Notwithstanding the possibility to establish further fundamental understanding of the influence of microbial population dynamics and physiological state on PHA production in these complex mixed culture systems, we have found ways in practice to improve the PAP performance of a biomass coming from PE2 by introducing an intervening conditioning step, to be applied to the harvested PE2 biomass, before the process element PE3. We refer to this intervening, conditioning step herein as acclimation and/or acclimation feast and famine conditioning.

An augmented PAP of a biomass coming from a PE2 can be achieved by subjecting the biomass within PE2 to a feast and famine selection strategy in conjunction with objectives and demands in the wastewater treatment. Selection refers to an environmental pressure that favors certain phenotypes of microorganisms to survive and become dominant in a bioprocess ecosystem over the course of a relatively long period of time. Feast and famine means that the biological treatment is carried out such that the biomass is disposed to alternating environments of available and scarcely available substrate in the form of RBCOD. During feast, biomass is provided with an excess of RBCOD and at least some of this assimilated RBCOD is converted into PHA. Under famine conditions with low RBCOD availability, the biomass may use the stored PHA in order to satisfy energy and carbon requirements for maintenance and growth. Such concepts of enrichment of a biomass with PAP are known to those trained in the art and have been previously described (WO 2011/073744A1 and WO 2012/023114A1).

Enrichment for PAP occurs due to the conditions of biomass selection (feast and famine as one possible but non-exclusive example) during the wastewater treatment process. Since biomass is growing and the surplus produced biomass is exported routinely as part of PE2, those populations of species of bacteria in the biomass that survive well due to the engineered environmental pressures of selection will tend to become more dominant populations of bacteria. Those populations of bacteria which do not grow well under the conditions of selection will become washed out of the process over time due to the routine of surplus produced biomass removal. Dominance of function or phenotype (PHA accumulation potential) is more important than dominance of any one species of bacteria in the process. Therefore the conditions of selection may result in an augmented PAP meaning a strong and steady presence of the PHA storing phenotype in the biomass but the populations of species representing that phenotypic behavior may be nevertheless changing over time. Operations of PE2 may be characterized in part based on a selected biomass or sludge retention time or SRT. The SRT is the average time a particle of biomass (or a given bacterial cell) is present in the bioprocess before it exits the bioprocess as surplus biomass.

Generally in wastewater treatment systems the biomass SRTs are in the order of days to weeks. The process of selection is typically over time frames that are multiples of the process SRT, namely, weeks to months. Therefore, selection is a gradual process of nurturing the presence of functionality in a biomass over a relatively long period of time wherein there may be several turn overs of the bacteria in the process because roughly after approximately every five SRTs one may consider that the original generations of bacteria in the biomass are effectively “washed-out” if they do not grow, or replaced by their growing descendants should they flourish. The disclosed acclimation conditioning is not considered to be a “selection” process because the biomass during acclimation is to be retained to the largest extent possible in the process, it is not a process performed with intent to grow the biomass, and the time frame of the acclimation process described herein may be measured by hours within a day.

In the PHA accumulation process of PE3, biomass with PAP is generally combined with RBCOD from PE1, whereby volatile fatty acids (VFAs) are a preferred type of RBCOD. In a well-controlled accumulation process, maximal biomass-PHA content is attained with a molecular weight preferably greater than 400 kDa (WO 2011/070544A2).

We have discovered a method of acclimation to be applied before disposing a biomass to conditions of a PE3 for enhancing the extant maximal capacity of PHA accumulation of surplus biomass from wastewater treatment (PE2). Methods that help to increase the PAP of a biomass improve the overall PE3 process productivity and thereby the economy of making a PHA-rich biomass and the subsequent PHA recovery from that biomass.

The intervening step of acclimation is embodied by subjecting at least some but preferably all of the biomass that is harvested from the biomass production step (PE2), to a limited number of fed-batch feast and famine cycles prior to subjecting the biomass to the accumulation process of PE3. In a preferred embodiment, acclimation is carried out in the same reactor as used for PE3. The accumulation process of PE3 is tantamount to subjecting the biomass to a process of prolonged feast. In the preferred embodiment, the acclimation cycles of feast-famine are accomplished using the same source of RBCOD to be used for the PE3 PHA accumulation. Since the biomass may be generally produced on a wastewater that is distinct from the substrate used for the accumulation process, the acclimation is a means to acclimate the biomass:

A. to the changed source of RBCOD before PHA production, and

B. to feast-famine operating conditions that are independent and distinct from PE2.

As such, a method of acclimation in advance of PHA accumulation (PE3) is further distinguished from a method or process to produce a biomass (PE2) with PHA accumulating potential due to a change of feedstock and the selection operating conditions used in the PE2.

Thus the RBCOD composition and/or water quality in the wastewater used to produce the biomass in PE2 may generally be distinguished in measureable character to the RBCOD composition and/or water quality used to acclimate the biomass and then produce PHA in the biomass (acclimation followed by PE3). The feast-famine of acclimation is also distinct from wastewater treatment in PE2 in that it is dynamically tuned in timing to the respiration response of the biomass rather than to the constraints of timing imposed by the flow rates in the wastewater treatment process of PE2. Notwithstanding the ability to purposefully integrate and engineer bioprocess principles of PAP enrichment in biological water treatment systems, the application of the acclimation is relevant even for a biomass that is harvested from wastewater treatment processes which have not been designed and built a priori, or generally modified and tuned a posteriori to accomplish or optimize the biomass PAP enrichment (Example 5).

The conditions of feast-famine in acclimation prior to PE3 are distinct from the conditions of selection in PE2 by feast-famine. Feast-famine selection in a PE2 is a means to produce a biomass with PAP. The process of selection is promoted due to an ongoing process of biomass harvesting. Those organisms that do not grow relatively well are washed out of the process in time. Therefore feast-famine selection requires an ongoing growth and removal of biomass over a relatively longer time in a PE2 than in the acclimation step. A biomass coming from PE2 that is to be used in a PE3 may exhibit a PAP but the full possible extent of the PAP this biomass can achieve may not be exhibited due to the history of the biomass in the PE2. Thus one may consider that the biomass is selected in PE2 to be rich in the PHA accumulating phenotype, but the extant physiological state of the biomass is not optimal with respect to achieving the best possible PHA accumulation response. Our findings of an acclimation step are applied in order to elicit an optimal or, at least, near optimal PHA accumulation response in the PE3.

Feast-famine cycles in acclimation are intended to ramp up the interpreted physiological state of the biomass towards a resultant maximal PHA accumulation response. Therefore, acclimation is not a method of biomass selection and production like a PE2; it is a method of biomass conditioning as a transitional step between a PE2 and a PE3.

The timing of repeatedly applied feast-famine cycles in acclimation is determined by the biomass response during the period of feast in each respective cycle. An onset of conditions of famine biomass respiration relative to a feast biomass respiration on RBCOD, generally occurs when RBCOD concentrations are negligible, and typically under 10 mg/L. Feast is stimulated by exposing the biomass, or fractions of the biomass at any given time, to an RBCOD concentration greater than 10 mg/L, but preferably greater than 100 mg/L, and less than 2000 mg/L. An elevated, feast, respiration rate may be measured, for example, in terms of an increased biomass oxygen demand.

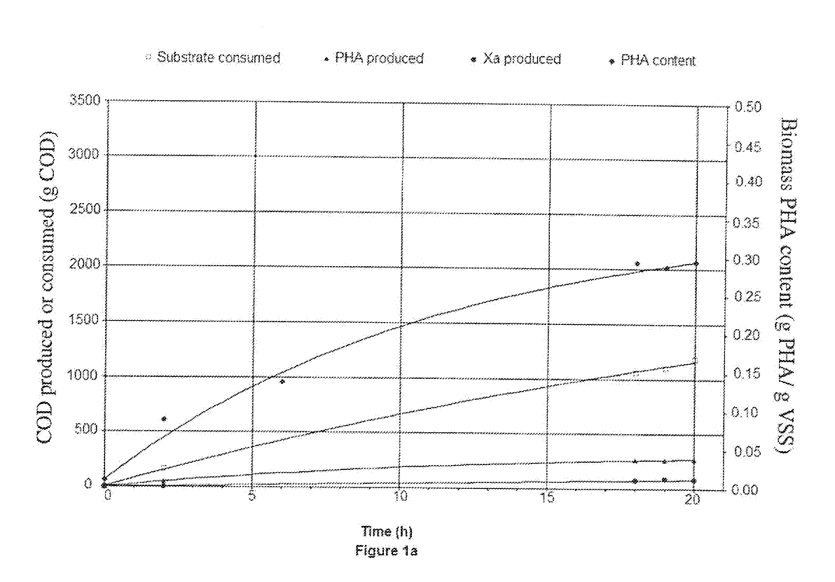

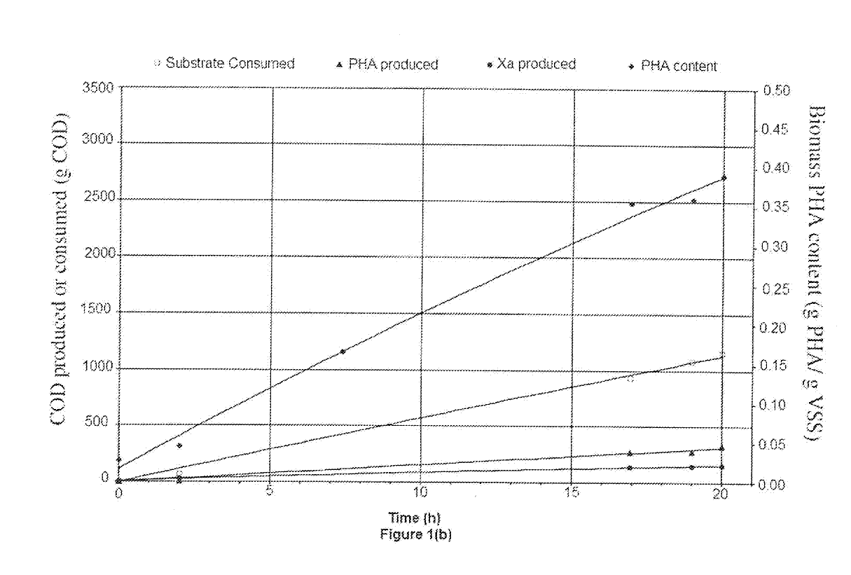

Samples of biomass respiring under conditions of famine, can be mixed with RBCOD to reach a range of maximum targeted initial RBCOD concentrations from 10 to 2000 mg-RBCOD/L. The maximum extant biomass oxygen uptake rate is observed as an asymptotic or maximum value reached with increasing initial RBCOD concentrations (see: WO2011/070544A2 FIG. 1). One may readily assess the respiration rate of biomass in a mixed liquor by the consumption rate of oxygen for aerobic processes and nitrate for anoxic processes. Dissolved oxygen or redox potential measurement devices can be applied, respectively for this purpose in aerobic or anoxic feast environments. One may reference the level of feast respiration rate achieved by a biomass in a mixed liquor to the maximum extant feast respiration rate that biomass could reach for any given source of RBCOD. A feast time may be defined as the time for which a biomass has been stimulated by contacting the biomass, or parts of the biomass at any given time, to RBCOD such that the biomass respiration rate is stimulated to be greater than 50% of the extant maximum respiration rate.

A change in aerobic respiration rate (or increase in oxygen demand) from famine respiration to feast respiration may be inferred by a decrease in mixed liquor dissolved oxygen concentration given constant conditions of aeration. Similarly an increase in oxygen demand is also inferred in measurement of an increased aeration air input that is necessary to keep a constant dissolved oxygen concentration in the mixed liquor.

Feast respiration may be sustained after a limited mass addition of RBCOD to the biomass due to the time it takes the biomass to process the mass of RBCOD that was added. Therefore, the biomass respiration rate may also be measured in terms of the rate of COD reduction in the mixed liquor. The feast time is therefore a period time after a limited mass addition of RBCOD is made to the mixed liquor and the RBCOD concentrations are generally greater than 10 mg/L. RBCOD concentrations may be more difficult to measure directly online but samplers exist for monitoring total organic carbon, or chemical oxygen demand. Relative changes in these parameters in time can be used to infer changes in RBCOD given known mass additions of RBCOD to a mixed liquor. Changes in RBCOD (addition and consumption) may also be inferred by indirect measurement methods such as spectroscopy, pH, and/or off gas analysis.

If the RBCOD contains volatile fatty acids then a decrease in pH will generally be observed due to a sudden acidic input to the mixed liquor. A subsequent increase of pH is associated with the consumption of these acids by the biomass in the mixed liquor. Thus by monitoring pH one can follow trends of addition and removal of RBCOD such as VFAs. Methods of spectroscopy rely on the measurement of light at selected wavelengths (visible, ultraviolet, infra-red, and/or near infra-red). For example, the addition of RBCOD with each pulse will result in PHA accumulation by the biomass and the PHA level will reach a maximum value when the RBCOD is consumed. The change of PHA in the biomass can be detected by adsorption of light in the infrared or near infrared wavelength ranges. Therefore the fate of RBCOD can be measured indirectly for each acclimation feast pulse by a signal that follows the trend of PHA in the biomass using spectroscopy. An increase in biomass respiration due to an RBCOD addition can also be characterized by an increase in production of respiration by-products such as carbon dioxide. Carbon dioxide concentrations can be measured in the off-gas that is produced due to the process aeration. Measurements of CO₂ concentrations in a gas are often made using infrared detectors. When the RBCOD has been consumed the respiration rate will decrease and the CO₂ concentrations in the off-gas will also decrease back to background levels. Therefore, the fate of RBCOD additions can be measured indirectly based on changes in off-gas CO₂ concentrations. These examples are provided without limitation to illustrate that knowledge of water chemistry and biomass respiration can be applied towards selecting methods that monitor for a trend in the response to repeated RBCOD additions made for the purposes of biomass acclimation conditioning. The duration and magnitude of these trends can be related to the rate and intensity of the biomass response. Relative changes of increase to the rate and intensity of the biomass response are indicative of a positive response of the biomass to a process of acclimation in advance of a PHA accumulation process.

Generally the first addition of RBCOD to the biomass provides a reference from which to assess subsequent feast responses from the biomass. This first and subsequent feast responses, as discussed above, can be quantified directly or indirectly in terms of a measure of the specific RBCOD removal rate, and/or a measure of the specific biomass respiration rate. The term “specific” is used to indicate that although these rates are measured volumetrically it is most preferable to compare these rates with respect to the amount of biomass per unit volume. Typically units of measurement may be g-COD/g-VSS/min or g-O₂/g-VSS/min, respectively, where VSS is volatile suspended solids, which is often used as a representative measure of the amount of biomass in the mixed liquor. In the use of indirect monitoring methods, the trend in measurement of a signal (S) or signals provides for quantitative estimation of a feast response that is proportional to the specific substrate removal rate and/or specific respiration rate. This maximum observed rate (expressed as “q”) for each cycle of feast in the acclimation process may be referred to as qₙ where n is the n^{th} feast-famine cycle. Therefore, q₁ is the above-mentioned measured first or reference feast response in the first feast-famine cycle for the biomass. Generally we find that the value of q increases with n where this increase is often observed to approach a limiting value asymptotically. Since acclimation takes time and resources, we anticipate that it may not always be practically or economically feasible to always run an acclimation towards achieving the maximum possible q from the biomass in advance of a PHA accumulation process. Notwithstanding, an objective of acclimation is to express an augmented PHA storage response from the biomass with a feedstock that is not the same as the one used to produce the biomass in the first place. A practical and measureable response of the biomass to the conditioning of the process and methods of acclimation is considered to be

$$\frac{{q}_{i}}{{q}_{1}} \geq 1.1 where i \geq 2$$

In another embodiment one could use the signal S towards estimating the duration of the feast per mg of RBCOD added per unit volume of reactor with units such as min/mg-RBCOD/L. Such a time could also be expressed as a specific time with respect to the amount of biomass per unit volume with units such as min/mg-RBCOD/mg-VSS. This specific duration expressed as “P” may be assessed for each feast in every acclimation feast-famine cycle, giving P₁, P₂ . . . Pᵢ . . . Pₙ. Analogous to the example involving the specific rates, a practical measureable response of the biomass to the conditioning of the process and methods of acclimation is considered to be

$$\frac{{P}_{i}}{{P}_{1}} \leq 0.9 where i \geq 2$$

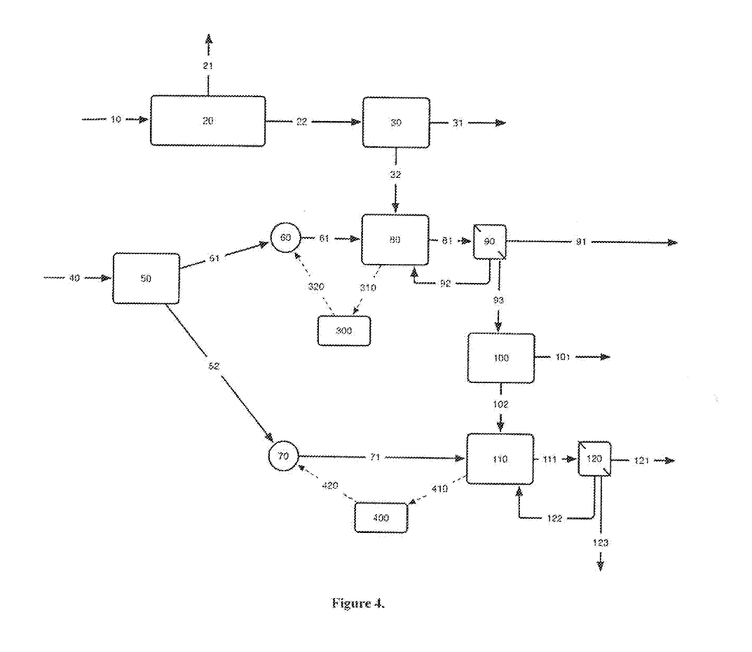

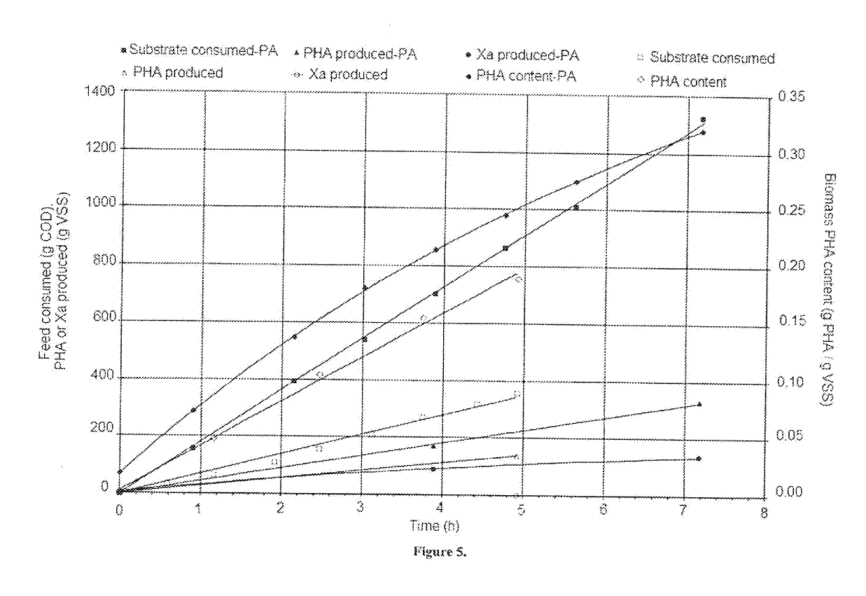

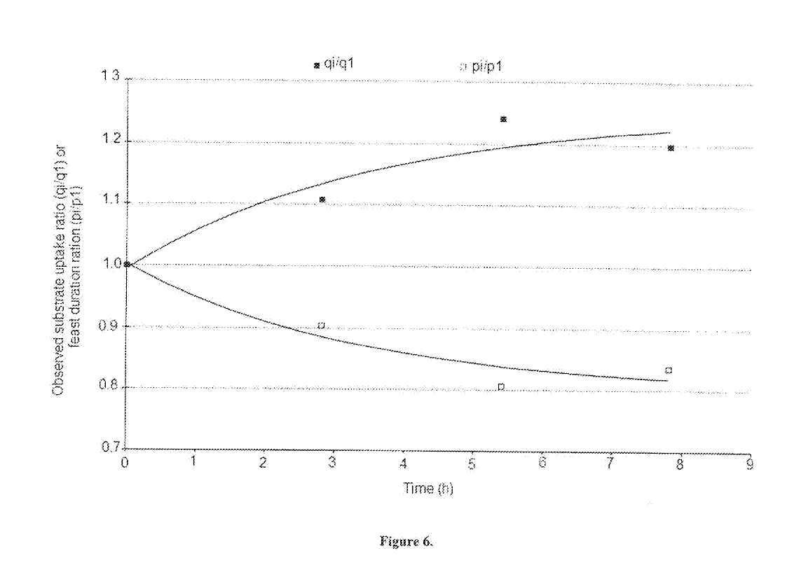

We have previously and repeatedly observed that a more active biomass will reach higher respiration rates when exposed to higher RBCOD concentrations, but generally RBCOD respiration stimulation concentrations greater than 100 mg-RBCOD/L will achieve a maximum or else near maximum respiration rate response (see: WO2012/023114A1 FIG. 6). Therefore, we often select a peak RBCOD stimulation concentration of 200 mg-RBCOD/L as a conservative target maximum concentration value to use when stimulating a biomass, or parts of a biomass at any given time, into a maximum or near maximum feast respiration response.

A period of famine is imposed after each respective feast stimulation during the acclimation process. After any given feast stimulation, the feast period of time is estimated from the measured period or interval of increase and decrease of biomass respiration rate, following the limited mass addition of RBCOD to the mixed liquor. The feast time is measured and a famine time may be applied as a multiple of the measured feast time before a next subsequent feast acclimation cycle that is stimulated again by a mass of RBCOD addition. For acclimation we consider that the applied famine time should be at least twice the preceding measured feast time, but preferably three times the preceding measured feast time. Therefore we define a feast to famine time ratio as being the estimated feast time for each feast stimulation (t_{fe}), divided by the imposed respective famine time (t_{fa}), wherein for the purposes of acclimation we consider that in the preferred embodiment for the i-th feast stimulation:

$${( \frac{{t}_{fe}}{{t}_{fa}} )}_{i} \leq 0.5 where i \geq 1$$

A response of improved PHA accumulation potential due to repeated feast-famine acclimation cycles may be measured in terms of any one or a combination of the following:
a relative decrease in the measured feast time per gram of RBCOD added per feast cycle,
a relative increase in the RBCOD uptake rate, and/or
a relative increase in the biomass specific respiration rate.

In a most preferred embodiment, PE3 is started without undue delay after the last applied acclimation feast and famine cycle. Notwithstanding, biomass may retain the effect of an improved accumulation response for some time after acclimation. In the shift from acclimation to accumulation process methods, the biomass may be thickened and dewatered, and otherwise maintained under conditions of famine pending the start of the PE3 PHA production.

It is not intended that the biomass is grown and removed during acclimation. In the preferred embodiment, the biomass harvested from a PE2 is retained in the acclimation process, and when acclimation is completed, the biomass is subject to conditions of a sustained biomass PHA accumulation in a PE3. Biomass can be retained during acclimation by methods such as:
Allowing for an increase in volume,
Periodic gravity settling (or dissolved air flotation or membrane separation) of the biomass, retention of the majority of the concentrated mixed liquor suspended solids, and removal of the supernatant with negligible or diluted suspended solids, or
Providing for a quiescent zone, whereby the majority of the suspended solids may be retained, while excess volume containing negligible suspended solids may be discharged.

Those specialized in the art of biological wastewater treatment will understand the concept of retaining a biomass in a process and will generally be versed in a suite of suitable methods based, for example, on principles of density difference and or physical exclusion to retain a biomass in a biological process. Generally, it is advantageous to retain the biomass during acclimation, since loss of biomass during the acclimation step, is a loss of biomass that is the resource intended to be exploited for PHA production in a PE3. Thus in a preferred embodiment, most of the biomass is retained in the acclimation process. Retaining most of the biomass means that 50% or more of the biomass is retained in the acclimation process. Related to this process feature is that the amount of biomass in the acclimation process does not increase by more than a fraction of the amount added to the process. Here the term “more than a fraction” means more than 25%.

Notwithstanding the possibility for new insights and interpretations in the future regarding the physiological state of a biomass with respect to a PHA accumulation process, we have discovered that feast-famine acclimation cycles with the same RBCOD to be used during accumulation, but different to the RBCOD used in any previous enrichment, increases the biomass extant PHA accumulation capacity. This increase in capacity is compared to that of the same biomass going directly from PE2 to PE3 without the benefit of any said acclimation conditioning.

Acclimation also enhances the extant substrate uptake and PHA storage rates of the biomass. Without limitation, three illustrative examples are given below in which the PAP of a biomass, which was produced with treatment of one distinct source of RBCOD during a wastewater treatment process (PE2), is shown to be improved significantly in a PE3 by applying methods of acclimation before a PE3 accumulation, wherein the RBCOD source for acclimation and accumulation steps are the same.

Examples of steps that may be included in methodologies utilizing the processes described herein and based on our experimental results, may include the following: A method or process of acclimation to increase the PHA accumulation response of a biomass that has been produced in a wastewater treatment wherein:
The RBCOD source for the acclimation method or process is different in some measureable character from the RBCOD source from which biomass was produced.
The RBCOD source for the acclimation method or process is the same as the one to be used for a subsequently performed PHA accumulation method or process.
The acclimation process is a fed-batch process whereby at least most of the biomass is retained in the process during acclimation, wherein
   Some form of separation method may be used to produce an effluent after or during selected feast and famine cycles, and
   Effluent is disposed from the process such that the majority of the biomass is retained per cycle, wherein more than 50 percent, preferably more than 70 percent and most preferably more than 90 percent of the biomass is retained per cycle.
The timing of the acclimation feast-famine cycles in the acclimation is determined by the biomass, extant metabolic activity whereby:
   The feast time is the time to remove more than 90 percent of the amount of RBCOD fed to the biomass during feast and
   The famine time is provided after feast and is controlled to be at least twice as long as the feast time, but preferably at least 3 times as long as the feast time.
The feast time may be assessed directly or indirectly by, for example, measurements of change in biomass respiration, pH, dissolved oxygen concentration, redox potential, off-gas analysis, spectroscopy, and/or soluble organic matter concentration.
The famine time may be defined as a contiguous period of time wherein the biomass or fractions thereof are maintained at a respiration rate that is less than 50% of the extant maximum potential and preferably less than 20%.
In the preferred embodiment, the number of acclimation cycles to be applied to the biomass prior to an accumulation process is determined by a measured trend in the biomass response to the repeated acclimation feast-famine cycles wherein:
   The process of acclimation is shorter than the SRT in the process used to produce the biomass,
   The number of acclimation feast-famine cycles is greater than one, preferably less than 40, and most preferably less than 20, and such that:
      Repeated feast-famine acclimation cycles are continued until the biomass feast specific substrate uptake rate (that is, the rate at which the biomass removes RBCOD from the water during a period of feast) in subsequent cycles exhibits an increase, that is at least more than a 10% increase, with respect to that of the feast specific substrate uptake rate during the feast period of the first feast-famine cycle, whereby most preferably,
      A sufficient number of acclimation cycles are applied in order to allow for the specific substrate uptake rate and/or specific respiration rate in repeated acclimation feast-famine cycles to approach a higher (maximum achievable) plateau value of specific substrate uptake rate and/or specific respiration rate, given that,
RBCOD is supplied to the biomass to achieve conditions of feast for each repeated acclimation feast-famine cycle, wherein:
   For each cycle, the biomass in the process reaches more than 50 percent of its extant respiration rate during feast,
   The biomass in the process is stimulated to feast by supplying substrate to the biomass as a whole or to at least some of the biomass at any given time during feast,
   The biomass is stimulated into feast with RBCOD such that the biomass or parts of the biomass at any given time are subject to a peak RBCOD concentration that is greater than at least 10 mg-COD/L, preferably greater than 100 mg/L and preferably, less than 2000 mg-COD/L.
In some embodiments, when biomass is stimulated into feast by applying substrate to a part of the biomass at any given time:
   Substrate containing RBCOD is continuously or semi-continuously re-circulated to at least one stimulation zone, whereby:
      A peak RBCOD concentration in the stimulation zone is greater than at least 10 mg-COD/L, preferably greater than 100 mg/L and preferably, less than 2000 mg-COD/L,
      The stimulation zone may be kept aerobic, anoxic or anaerobic with respect to measurements of redox potential,
      The stimulation zone(s) is (are) distinguishable from the remaining process volume, where most of the rest of the biomass is maintained, by virtue of some form of separating structure, and
   Apart from any stimulation zone volume(s) used in the process, the biomass is maintained and subjected to repeated cycles of feast and famine respiration under conditions that are aerobic or anoxic with respect to redox potential.
After the last acclimation feast-famine cycle, the biomass are disposed to conditions of PHA accumulation with continuous or semi-continuous supply that includes the same acclimation RBCOD feedstock and with organic loading rate provided on demand in order to sustain a high (feast) respiration rate, and a sustained accumulation of PHA in the biomass.

### Example 1: Enhancement of PHA Accumulation Potential by Applying an Intermediary Step of Feast-Famine Acclimation on a Biomass Harvested from a Wastewater Treatment Process

A series of feed-on-demand PHA accumulations were conducted in a fed-batch pilot-scale process using acetate as substrate and activated sludge biomass. The biomass was made to be enriched with PAP from the RBCOD of a municipal wastewater, which generally contained low levels of acetate (<10% soluble COD). Before the accumulation process, an intervening acclimation step was imposed whereby the biomass was exposed to a number of feast-famine cycles wherein the famine time targeted a defined feast-famine ratio. The RBCOD for the acclimation was acetate (>99% soluble COD), and the acclimation was found to significantly improve the PAP of the biomass when compared to the same accumulation process without any intervening step of acclimation.

Three PHA accumulation production runs were conducted under aerobic conditions with acetate as RBCOD (84 g COD/L) and an activated sludge biomass as input raw materials to the PE3. The biomass was produced at pilot scale from treating the RBCOD of a municipal wastewater as previously described (WO 2012/023114A1; and Morgan-Sagastume F, Valentino F, Hjort M, Cirne D, Karabegovic L, Gerardin F, Johansson P, Karlsson A, Magnusson P, Alexandersson T, Bengtsson S, Majone M, Werker A. Polyhydroxyalkanoate (PHA) production from sludge and municipal wastewater treatment, Water Science and Technology, 69.1, 177-184, 2014).

A first accumulation production run was conducted without any nutrient (N and P) addition. The second accumulation run was conducted with nutrient addition to limiting levels, and the third accumulation run was conducted with nutrient addition to limiting levels and an intervening acclimation step.

In the accumulation production runs with nutrient addition, measured amounts of NH₄Cl and KH₂PO₄ were added to the acetate accumulation feedstock so as to reach a COD:N:P mass ratio of 100:1.2:0.07. The feed pH was adjusted to 5 with the addition of NaOH.

In the third accumulation run, the acclimation consisted of two feast-famine cycles without biomass wasting and with an imposed feast-to-famine ratio of 0.33. The duration of the feast-famine cycles was defined based on the target feast-to-famine ratio of 0.33 wherein the famine time was let to be three times as long as the time taken for the biomass in the feast. Feast conditions were created by rapid substrate additions so as to reach a peak RBCOD concentration of 100 mg COD/L. The start of feast respiration response was indicated by a decrease in dissolved oxygen levels given constant aeration. The duration of the feast was therefore indicated by the time interval from decrease and increase in the dissolved oxygen concentration. The increase in dissolved oxygen level is indicative of a decrease in the biomass respiration rate when the added mass of RBCOD becomes assimilated by the biomass.

The three PHA accumulation production runs were conducted under similar conditions in a fed-batch process using a feed-on-demand respirometric control (WO 2011/070544A2). The first two accumulation runs lasted 20 h each, while the accumulation with acclimation lasted in total 23.8 h, including 7.3 h of acclimation and 16.5 h of accumulation. The initial conditions for the three accumulation runs are summarized in Table 1.

The accumulation production runs were conducted with cumulated harvested activated sludge biomass from the pilot municipal wastewater treatment facility. Harvesting of biomass was over no more than six days before the accumulation. The harvested biomass was aerated pending the accumulation run. The initial biomass concentrations in the accumulation runs were within 0.5-0.8 g VSS/L, which were achieved by diluting with tap water up to 400 L. The temperature during the accumulation was not controlled and ranged from 23° to 33° C. Dissolved oxygen (DO) and pH were monitored with online probes. The reactor mixed liquor was sampled (600 mL) at selected time points. For the beginning (t=0) and end samples, the TSS and VSS concentrations were determined using standard methods (APHA, 2005). Part of each 600 mL grab sample was centrifuged (3200×g for 5 min), and the supernatant was further filtered (1.5 μm; Whatman) and analyzed for soluble COD (LCK 114, 314, and 614), total N (LCK 138, 238, and 338) and total P (LCK 348) using Hach-Lange kits. The biomass pellet after centrifugation was dried (105° C. for 24 h) and analyzed for ash and PHA contents based on thermal gravimetric analysis (TGA), as previously reported (WO 2012/022998A1). The three PHA accumulations were conducted over a relatively short period of 3 weeks during stable operation of the wastewater treatment pilot plant.

For the purpose of these assessments, we have defined active biomass (Xa) as the mass of biomass, measured as volatile suspended solids (VSS), minus the mass of PHA in the VSS.

Acclimation as an intervening step to biomass production and PHA accumulation processes was found to substantially increase the biomass PHA accumulation potential (PAP) and the total amount of polymer produced during the accumulation compared to those achieved in accumulation runs without the benefit of any acclimation conditioning of the biomass (FIG. 1). In the first accumulation without N and P addition and without acclimation, an increase in biomass PHA content was observed up to a saturation level of 0.30 g PHA/g VSS after 20 h, corresponding to 0.46 grams of PHA produced per gram of initial active biomass (g PHA/g Xaᵢ), which became the benchmark or reference extant level for this biomass. Nutrient addition in the feed without acclimation was observed to result in an increase of PAP up to 0.39 g PHA/g VSS after 20 h, corresponding to 0.85 g PHA/g Xaᵢ. Finally, with the benefit of nutrient addition in the feedstock and acclimation a much higher PAP of 0.46 g PHA/g VSS was achieved with 2.18 g PHA/g Xaᵢ, after 16.5 h.

Both the overall substrate utilization rates and PHA production rates or productivities increased 2- and 4-fold with the acclimation step (Table 2) compared to those without acclimation. Nutrients were found to have an important positive effect on the biomass performance in PAP, and this enhancement became even more augmented with the benefit of the acclimation. Even given the time required to condition the biomass with the acclimation step, the overall PHA productivity of the accumulation process was found to be substantially increased (Table 2). This increase in PHA productivity was interpreted to be associated with a stimulated increase in active biomass yields and rates.

In terms of active biomass growth, whereas only 41 g VSS were produced during the first accumulation run without nutrient addition, 78 g VSS were produced during the second accumulation run with added nutrients, and 370 g VSS were produced with added nutrients and acclimation.

In conclusion, we found that the PAP of a biomass harvested from a municipal wastewater treatment process could be enhanced by applying an acclimation step consisting of just two aerobic feast-famine cycles. The feast-famine cycle times were dynamically controlled based on the biomass respiration rate and the RBCOD source was distinct from the wastewater treatment process and the same as the one used for the PHA accumulation process.

### Example 2: Enhancement of PHA Accumulation Potential by Applying an Intermediary Step of Acclimation on a Biomass Harvested from a Wastewater Treatment Process

Two PHA accumulation runs were undertaken in a fed-batch laboratory-scale reactor using a mixture of acetate and propionate as RBCOD and an activated sludge biomass enriched for PAP in the course of the treatment of a synthetic municipal wastewater whose acetate content was less than 15% of the total wastewater COD and without propionate. An acclimation step was applied prior to one of the accumulation runs whereby the biomass was exposed to feast-famine cycles targeting a pre-determined cycle duration. We found that the inclusion of acclimation before the process of PHA accumulation significantly enhanced the PAP of the biomass. The accumulation runs were performed by means of two successive RBCOD pulse additions.

The activated sludge biomass used in the accumulations with and without acclimation was enriched for PAP under feast-famine conditions in a laboratory-scale sequencing batch reactor (SBR) with a 1 L working volume. The SBR was inoculated with activated sludge from the Roma North wastewater treatment plant and operated with an organic loading rate of 3.1 g COD/L/d and hydraulic and sludge retention times of 2.14 h and 1.5 d, respectively. The reactor was aerated by membrane compressors and mixed mechanically (250 rpm). Temperature was controlled at 22.5° C. with a water jacket and pH remained at 8.0 without the need of pH-control. For the wastewater treatment, SBR cycles lasted 1.5 h (16 cycles per day), including feeding (2.5 min, aeration and mixing), reaction (59.5 min, aeration and mixing), settling (20 min) and effluent discharge under quiescent conditions (7.5 min). Biomass discharge (0.5 min, mixing) was conducted during 6 cycles of the total 16 cycles per day. The feed consisted of a mixture of soluble compounds that are known to represent the soluble fraction of municipal wastewater (275 mg COD/L): acetic acid (11% of the total COD), yeast extract (13%), glucose (27%), starch (25%), peptone (24%), NH₄Cl (35 mg N—NH₄⁺/L), and KH₂PO₄ (3 mg P—PO₄³—/L). The activated sludge biomass was harvested for PHA production at the end of the famine phase of different cycles during a period of stable operation of the SBR. Such stable operating conditions of the biomass response were achieved already after 15 days of operation.

Two fed-batch PHA accumulation runs were conducted aerobically (DO=8 mg/L) at the same temperature and pH as the SBR. In both accumulation runs, the pH was controlled at 8.0 with H₂SO₄ 0.8 M additions and RBCOD was provided as a mixture of acetate (85% of total COD) and propionate (15% of total COD) in two pulse additions, one at the beginning and the other after approximately 7 hours from the start of each accumulation. A food-to-microorganism ratio of 2 g COD/g VSS was targeted with the first substrate pulse addition at the beginning of both accumulations. The accumulation process mixed liquor biomass was sampled at selected time points for monitoring changes in biomass and water quality.

A first accumulation run was conducted over 24 h in a 0.5-L reactor. Harvested biomass was diluted to an initial concentration of 0.5 g VSS/L with tap water and 0.2 L of mineral medium (2 mg FeCl₃.6H₂O/L, 3 mg Na₂EDTA/L, 330 mg K₂HPO₄/L, 260 mg KH₂PO₄/L, 100 MgSO₄.7H₂O/L, 50 mg CaCl₂.2H₂O/L, 0.1 mg ZnSO₄.7H₂O/L, 0.03 mg MnCl₂.4H₂O/L, 0.3 mg H₃BO₃/L, 0.2 mg CoCl₂.6H₂O/L, 0.01 mg CuCl₂.2H₂O/L, 0.02 mg NiCl₂.H₂O/L, 0.03 mg Na₂MoO₄.2H₂O/L. 20 mg thiourea/L). The substrate pulse additions targeted 1.0 g COD/L in the reactor and contained no nutrients. However, nutrients were provided in excess at the beginning of the accumulation targeting a COD:N:P ratio of 100:10:5 in the reactor, with PO₄³⁻ provided in the mineral medium and NH₄Cl as a direct addition just before the initial RBCOD pulse.

In a second accumulation run, the biomass was first disposed to an acclimation step of 6 h prior to the PHA accumulation process of 18 h. The acclimation step and accumulation run were both conducted in the same vessel. The day before the acclimation, biomass harvesting in each SBR cycle was performed so as to maintain the biomass concentration around 1.0 g VSS/L. The acclimation consisted of 4 feast-famine cycles with a defined, constant cycle duration of 1.5 h and without biomass wasting. During the acclimation, the biomass was subjected to feast by additions of an acetate-propionate mixture with nutrients in excess (COD:N:P=100:12.7:1) as to reach up to a maximum concentration of 200 mg COD/L in the reactor per feast event. Before starting the accumulation run, the biomass was retained by settling in the reactor, and the effluent supernatant was discharged. The vessel liquid volume was re-established to 1 L by adding 0.4 L of the same mineral medium described above and tap water. The substrate for the acclimation and accumulation had the same acetate-propionate composition but without nutrients, and the substrate input spikes targeted 2.0 g COD/L in the reactor in order to have the same food-to-microorganism as in the first accumulation. Nutrients were provided in excess at the beginning of the accumulation targeting a COD:N:P ratio of 100:5:2.5 in the reactor, with PO₄³⁻ provided in the mineral medium and NH₄Cl as a direct addition just before the initial substrate spiking. No more nutrient addition was performed during the accumulation.

Acetate and propionate were analyzed from filtered samples (0.45 μm) by gas chromatography (stationary phase Carbowax 20 mol L⁻¹ 4% on CarboPack B-DA; Perkin-Elmer 8410). Ammonium N was also determined from filtered samples by Nessler spectrophotometry (Shimadzu UV-Visible 1800 Spectrophotometer), in which the absorbance at 420 nm of the final complex NH₂Hg₂I₃ was proportional to its concentration and to that of NH₄⁺.

For PHA determination, mixed liquor samples were first treated with a small amount of NaClO solution (7% active Cl₂, diluted 1 in 5) in order to inhibit biological activity and then stored at −4° C. Before the analysis, PHA samples were thawed and centrifuged (10000 rpm, 30 min) in order to discharge the supernatant. The pellets were treated by adding 2.0 mL of methanol containing 3% (v/v) H₂SO₄, and 1.0 mL of chloroform in a screw-capped test tube and were then kept at 100° C. for 60 min for mild acid esterification. After cooling to room temperature, 1.0 mL distilled water was added and the whole sample was shaken for 10 min. After separation of the two phases, 1.0 μL of the organic phase was injected for GC analysis (stationary phase 2% Reoplex 400 on Chromosorb GAW 60 to 80 mesh, 8-ft column). The relative abundances of 3-HB and 3-HV monomers were quantified by using the commercial co-polymer poly-3-hydroxybutyric acid-co-3-hydroxyvaleric acid (PHBV) of known 3-HV content (5%) as standard (Sigma-Aldrich).

The active biomass (Xa) levels were calculated as the difference between the mixed liquor VSS and PHA contents. The following COD conversion factor were used: 1.42 g COD/g Xa from the generic heterotrophic biomass formula C₅H₇O₂N, 1.67 g COD/g 3-HB, 1.92 g COD/g 3-HV, 1.067 g COD/g acetic acid and 1.51 g COD/g propionic acid.

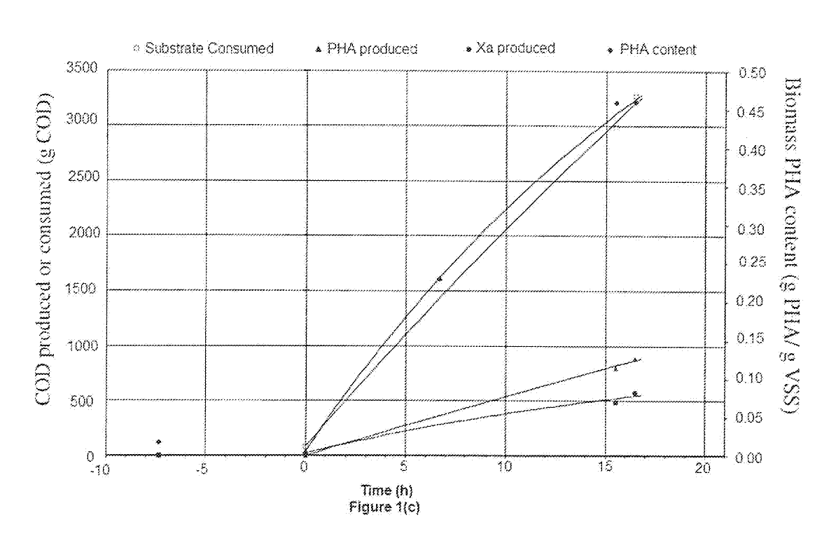

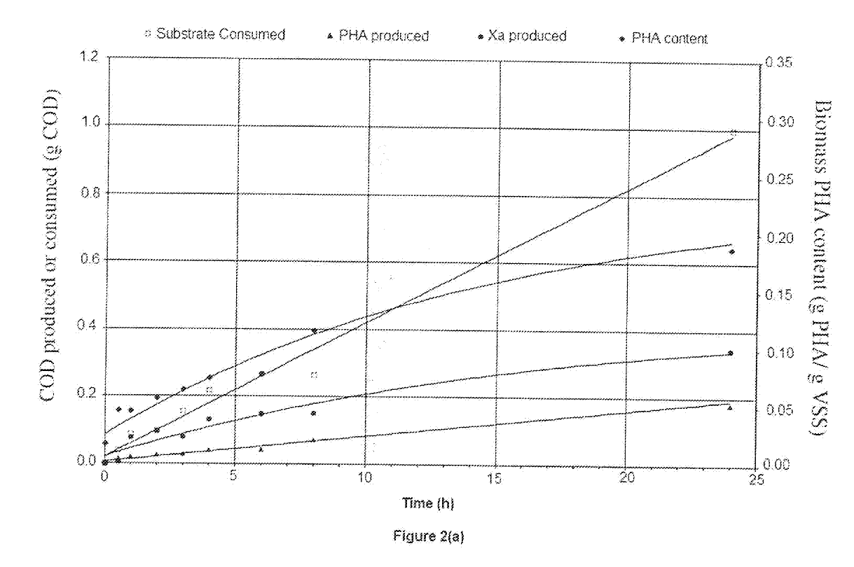

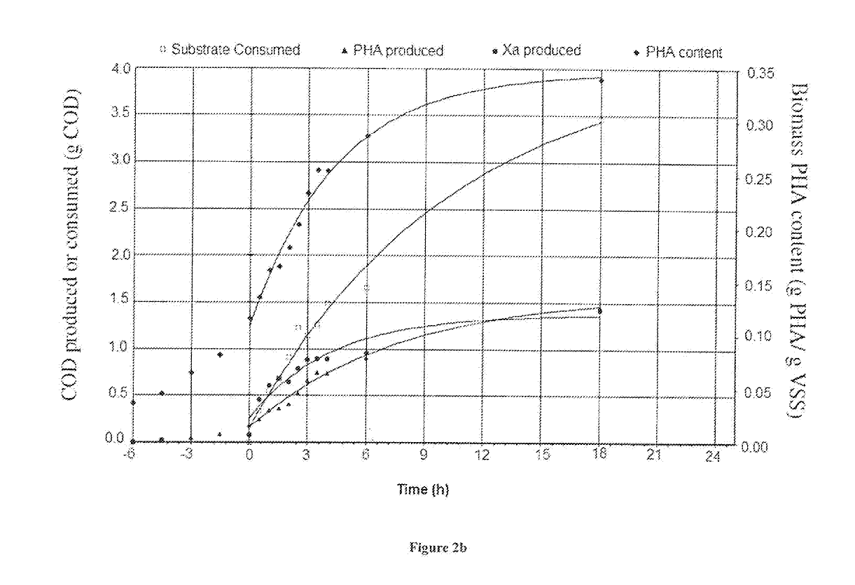

The acclimation of 4 feast-famine cycles (6 h) was found to enhance substantially the PHA storage capacity of the biomass with respect to the case without any acclimation. With the acclimation, the biomass PHA content during the accumulation increased 1.9 fold from 0.18 to 0.34 g PHA/g VSS (FIG. 2) and the productivities increased 2.2 fold from 0.6 to 1.25 g PHA/g Xaᵢ. Similarly to Example 1, the acclimation was observed to promote higher PHA production productivity and this was interpreted to be due to an increase in substrate utilization rates (from 130 to 280 g COD/g Xaᵢ/h) and PHA accumulation rates (from 50 to 100 mg PHA/g Xaᵢ/h) based on an overall process time of 24 h.

Additionally, we found that the acclimation promoted an increase in PHA storage yield (YΔPHA/ΔS) from 0.19 to 0.34 g COD/g COD with similar active biomass yields (Y_{ΔXa/ΔS}) of 0.48 and 0.44 g COD_{Xa}/g COD. During the accumulation runs, acetate and propionate were consumed simultaneously but at different rates. Due to the availability of nutrients in excess, both PHA storage and active biomass growth occurred simultaneously as demonstrated by the increase in PHA and active biomass over time (FIG. 2). Active biomass growth was considered to have further supported the PHA productivity results.

During the acclimation, PHA was accumulated as a result of a cumulative increase in biomass PHA content with negligible active biomass growth, (FIG. 2b). The biomass PHA content increased from 0.04 to 0.14 g PHA/g VSS, which was reflected in a VSS concentration increase from 970 to 1240 mg/L. The acetate-propionate substrate provided during the feast phases was completely removed. The PHA accumulation after each feast-famine cycle suggested a too short famine phase because generally the stored PHA is expected to be consumed during the following famine. Nevertheless, we found that a sufficient famine period can be ensured by dynamically tuning the acclimation feast-famine cycle time as in Example 1. Notwithstanding the accumulated PHA due to a fixed feast-famine cycle time, the acclimation step did stimulate an improved storage response in the biomass, which served to enhance PHA production during the accumulation.

In conclusion, we found that the acclimation, consisting of four feast-famine cycles with the same substrate used for the accumulation, but different to that used for biomass production, led to increased biomass PHA contents and PHA productivities as an interpreted direct consequence of higher substrate uptake and PHA storage kinetics.

### Example 3: Evaluation of Acclimation Effects on Stimulating PHA Storage Potential on Biomass Harvested from a Wastewater Treatment Process

Activated sludge biomass previously enriched for PHA-accumulation potential during the treatment of synthetic municipal wastewater was subjected to a series of 16 feast-famine cycles (1.5 h per cycle) without biomass wasting over 24 h. The activated sludge biomass had been previously enriched in PAP in the same manner as described in Example 2, and the 16 feast-famine cycles were applied in the same SBR, as described in Example 2, except that the feed was shifted from the VFA-poor municipal wastewater to the mixture of acetate and propionate with nutrients in excess (COD:N:P=100:12.7:1). Before starting the acclimation feast-famine cycles, the biomass was settled and supernatant effluent was discharged. Substrate feeding and analyses for ammonium, VSS, and biomass PHA content were also performed as described in Example 2.

The effect of the acclimation series of time-defined feast-famine cycles on biomass storage response was evaluated based on the change in the ratio of the duration of the feast to the total aerobic time, and the specific PHA storage and substrate consumption rates of the active biomass during the feast (FIG. 3). The application of a first feast-famine cycle with the acetate-propionate mixture led to an increase in the feast-to-aerobic-time ratio from 0.08, as recorded with synthetic municipal wastewater in the SBR, to 0.38. However, this ratio decreased after the second cycle and remained thereafter relatively constant around 0.30 (FIG. 3A). A 6 to 7 fold increase in specific PHA storage rates was observed after the second cycle, but consistently after the fourth cycle (FIG. 3A) with respect to that of the first cycle. The increase in storage rates was accompanied by a 1.2-fold increase in RBCOD consumption rates after the first cycle. PHA tended to accumulate over the cycles, but active biomass started to accumulate only after the fourth cycle (FIG. 3B).

In conclusion, by subjecting the biomass to two but especially four feast-famine cycles with the RBCOD to be used for PHA production, a quick physiological adaptation of the biomass was found to occur and this resulted in a sustained substantial increase in specific PHA storage rates.

TABLE 1
Summary of characteristics of the initial mixed liquor biomass before
starting the experiments in each accumulation run

| | Accumulation | Accumulation | Accumulation |
|---|---|---|---|
| | run 1 | run 2 | run 3 |
| | (No N & P | (N & P | (N & P |
| | addition, no | addition, no | addition, |
| Parameter | acclimation) | acclimation) | acclimation) |
| Active biomass | 0.84 | 0.52 | 0.59 |
| (Xa, g/L) | | | |
| Ash solids fraction | 0.32 | 0.52 | 0.40 |
| (g/g TSS) | | | |
| Soluble total N (mg/L) | 13 | 9 | 26 |
| Soluble total P (mg/L) | Not measured | 0.9 | 2.2 |
| Biomass PHA content | 0.01 | 0.01 | 0.01 |
| (g PHA/g VSS) | | | |

TABLE 2
Overall yields and rates during the PHA accumulation runs

| | Accumulation | Accumulation | Accumulation |
|---|---|---|---|
| | run 1 | run 2 | run 3 |
| | (No N & P | (N & P | (N & P |
| | addition, no | addition, no | addition, |
| Parameter | acclimation) | acclimation) | acclimation) |
| PHA storage yield | 0.28 | 0.25 | 0.26 |
| (Y_{ΔPHA/ΔS}, COD/ | | | |
| gCOD) | | | |
| Biomass yield | 0.07 | 0.10 | 0.16 |
| (Y_{ΔXa/ΔS,} gCOD/ | | | |
| gCOD) | | | |
| Substrate utilization | 0.14 | 0.28 | 0.58 |
| rate (gCOD/gXaᵢ/h) | | | |
| PHA accumulation | 23 | 43 | 91 |
| rate (mgPHA/gXaᵢ/h) | | | |
| Biomass growth | 6 | 19 | 66 |
| rate (μ, mgXa/ | | | |
| gXaᵢ/h) | | | |

Conversion factors: gCOD/gAc = 1.0667; gCOD/gXa = 1.45; gCOD/gPHA = 1.67
Rates were calculated considering the total time as the addition of acclimation and accumulation times

### Example 4: Process Schematic for Expressing Enhanced PHA Accumulation Potential with an Intermediary Step of Acclimation

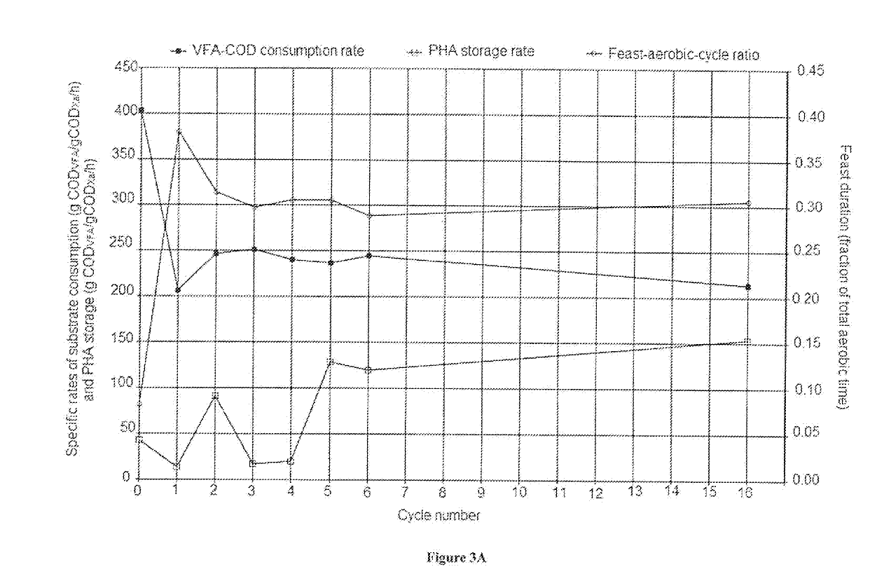

FIG. 4 provides an example of a process schematic diagram that illustrates practical steps of a process in the implementation of a preferred embodiment. A process water or wastewater (10) is ameliorated in a biological treatment process producing effluent streams of the process water or wastewater with improved water quality (21) and a surplus biomass (22). The mixed liquor containing the surplus biomass (22) may be directed to a temporary storage (30) wherein the period of storage may be also utilized to achieve goals of extended dewatering to produce a dewatering effluent (31) and a source of concentrated biomass for PHA production (32). During storage, the biomass activity is maintained. In some embodiments, some form of aeration occurs during storage to aid in maintaining biomass activity. In some embodiments, nitrate is added for at least part of the time of the biomass is in storage 30.

This mixed liquor containing surplus biomass (32) is then directed to processes and methods of feast-famine acclimation (80) followed by PHA accumulation (110). Acclimation and PHA accumulation may be performed apart in separate tanks and volumes as depicted in FIG. 4, or else both objectives could be similarly achieved sequentially within the same tanks and volumes. This is to say that the methods and processes surrounding 80 and 110 may be engineered to be specialized to the distinct tasks of acclimation and PHA accumulation, respectively. Alternatively, they may both be engineered to serve combined tasks of acclimation and PHA accumulation. Acclimation and PHA accumulation may be on the same site as the biological treatment (20), or the biomass (32) that is produced in (20) may be transported to another location for the PHA production.

For acclimation and PHA accumulation a feedstock of RBCOD is supplied (40) and, if necessary, a holding volume or equalization tank (50) can serve as a reservoir of influent RBCOD for both acclimation and PHA accumulation methods and processes. Generally influent (40) is distinct in some measure of the water quality from the influent (10) that was used to produce the surplus biomass (22).

The RBCOD feedstock influent (51) is pumped (60) to mixed liquor containing biomass that has been disposed (32) to the batch acclimation process. The input of a limited mass of RBCOD (61) to process (80) generates a feast response in the biomass and this response is monitored (310) and a controller (300) determines the feast time. Given a measured feast time, a famine time is applied whereby the famine time is a multiple greater than 2 of the feast time. At the end of the famine time the controller (300) initiates (320) another cycle of acclimation feast-famine by instructing the pump (60) to provide a repeated limited mass input of RBCOD to the biomass. These repeated cycles of acclimation feast-famine are continue until a criterion of increased biomass feast response, such as a significant decrease of the feast time is monitored, or else a maximum number of cycles has been reached. At least two acclimation feast-famine cycles are applied.

During acclimation, biomass is generally retained in the process (90), such that excess volume may be discharged (81) and the biomass is separated (90), returned (92), and retained in the acclimation process (80). Effluent from acclimation (91) will generally be with significantly reduced concentration of RBCOD in comparison to the process influent (61). Acclimation is generally carried out under either anoxic or aerobic conditions during feast and for at least part of the time during famine.

After a batch of biomass disposed from (30) has been subject to the methods and process of acclimation, it is directed (93) to methods and process of sustained PHA accumulation (110). Some form of temporary storage (100) of acclimated biomass may be provided for, and during storage a greater level of separation of the biomass from the mixed liquor may be accomplished producing an effluent (101) and a source of concentrated acclimated biomass (102). During storage the biomass activity is maintained. Storage may generally include some form of aeration. In some embodiments, nitrate is added for at least part of the time of the biomass storage in 100.

The PHA accumulation process is a batch process wherein acclimated biomass is subjected to a period of continuous feast without any intervening periods of famine. PHA accumulation is generally carried out under either anoxic, or aerobic conditions for sustained intervals for at least part of the time. The biomass respiration is monitored (410) and interpreted by a controller (400) that determines the volumetric flow rate of feedstock (52) pumping (70) from the RBCOD reservoir (50) to the PHA accumulation process (110). RBCOD influent (71) is supplied to the accumulation process so as to stimulate, on average, a sustained near maximal extant biomass respiration rate. A feed-on-demand control (400) of pumping rate is to be achieved wherein the biomass is maintained with a near maximal respiration rate such that the pumping rate (70), on average, just matches the biomass capacity in rate to remove RBCOD from the influent feedstock.

During PHA accumulation, biomass is generally retained in the process (120), such that excess volume may be discharged (121) and the biomass is separated (120), returned (122) and retained in the accumulation process (110). Effluent from PHA accumulation (121) will generally be with significantly reduced concentration of RBCOD in comparison to the process influent (71).

Biomass with significant PHA content may be harvested (123) during and at the end of the fed-batch process. The PHA-rich-biomass that is produced by the accumulation process is disposed to steps of further dewatering and the PHA may be recovered from the biomass as a value-added product.

We have found that the amount of PHA a surplus biomass (22) can accumulate in a PHA production process (110) is increased significantly by subjecting the biomass to an intervening acclimation process (80).

### Example 5: Increased Feast Response and Enhancement of PHA Accumulation Potential by Applying a Feast-Famine Acclimation to Biomass Harvested from a Wastewater Treatment Process

Two PHA accumulation runs were conducted in a pilot-scale process with biomass separation and recirculation using fermented waste-sludge liquor as RBCOD source and activated sludge biomass from a full-scale municipal wastewater treatment plant as input raw materials. The activated sludge biomass was harvested from a municipal wastewater treatment process that was not designed and built a priori to accomplish PHA accumulation potential (PAP enrichment), but that was nevertheless conducive to some measure of PAP enrichment by nature its process operation. An acclimation step was applied prior to one of the accumulation runs whereby the biomass was exposed to feast-famine cycles targeting a defined feast-famine ratio for a defined number of cycles. The RBCOD for the acclimation and accumulations was mostly VFAs (>90% of soluble COD) present in the liquor of fermented waste sludge. The RBCOD feedstock for the acclimation and PHA accumulation processes was distinct from the water quality of the full-scale influent municipal wastewater from which the biomass was grown. The inclusion of acclimation before the process of PHA accumulation significantly enhanced the biomass response to the new feedstock and resulted in an increased level of PHA accumulation when compared to the same accumulation process with the same source of biomass and RBCOD feedsock but without the benefit of the intervening acclimation step.

Two PHA accumulation runs were conducted under aerobic conditions in a pilot-scale process previously described (Morgan-Sagastume F, Hjort M, Cirne D, Gérardin F, Lacroix S, Gaval G, Karabegovic L, Alexandersson T, Johansson P, Karlsson A, Bengtsson S, Arcos-Hernández M V, Magnusson P, Werker A 2015. Integrated production of polyhydroxyalkanoates (PHAs) with municipal wastewater treatment at pilot scale. Bioresource Technology, 181, 78-89) and consisting of a continuously-fed, well-mixed reactor followed by a settler for biomass separation and recirculation. The first accumulation run was conducted without an acclimation step, but an acclimation step was applied prior to the second accumulation run. The RBCOD for both accumulation runs and the acclimation step consisted mostly of VFAs (>90% of soluble COD) present in the liquor of fermented waste sludge used as substrate feedstock. The fermented waste-sludge liquor was produced at pilot scale in a batch fermenter fed with thickened waste activated sludge (3-5% w/w TS) followed by a centrifugation unit for solids separation in the same pilot-scale system referenced above. The biomass used in the accumulation runs was harvested from a full-scale activated sludge process treating municipal wastewater with biological nitrogen removal; therefore, the RBCOD used in the accumulations and acclimation was different to the RBCOD treated by the biomass in the full-scale plant. The time elapsed from harvesting the biomass to starting the first accumulation run was 24 h including 1.5 h pre-aeration, and to starting the second accumulation run was 37 h including 9 h pre-aeration.

FeCl₃ was added to the centrate from fermented waste sludge produced in the pilot plant to precipitate PO₄³⁺—P, and a COD:N:P mass ratio of 100:12:0.005 was obtained in the fermented waste-sludge liquor used as accumulation feedstock. The pH of the fermented sludge liquor was 5.1.

The two PHA accumulation runs were conducted under similar conditions in a fed-batch process using a feed-on-demand respirometric control (WO 2011/070544A2). The first accumulation run lasted 4.9 h, and the second accumulation run lasted in total 16.2 h, including 9 h of acclimation and 7.2 h of accumulation. In the second accumulation run, the acclimation consisted of a pre-defined number of four feast-famine cycles without biomass wasting and with an imposed acclimation feast-to-famine ratio of 0.25. The acclimation feast-famine cycles were operated similarly as described in Example 1. The initial biomass concentrations in the accumulation runs were 1.5 and 1.30 g VSS/L for the first and second run, respectively. The temperature during the accumulation runs was constant and controlled at 25° C. The sampling and analytical techniques used were the same as specified in Example 1.

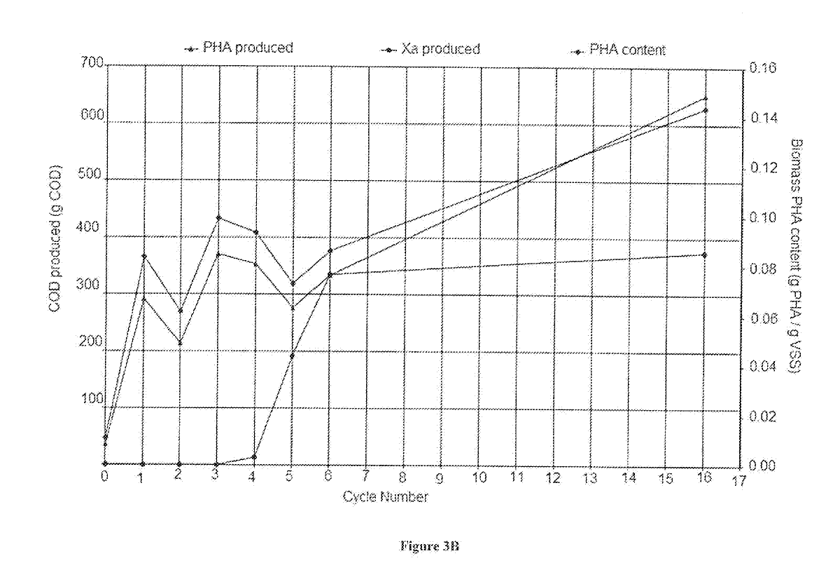

Similarly as in Example 1, the acclimation step in this case was found to increase the biomass PHA content and the total amount of PHA produced during the initial phase of the accumulation process compared to those achieved in the accumulation run made without benefit of an intervening step of acclimation (FIG. 5). In the accumulation with an acclimation step, the biomass PHA content was 30% higher (0.25 vs 0.19 g PHA/g VSS) and the amount of PHA produced was 60% higher (222 vs 138 g PHA) than those measured in the accumulation without acclimation after 4.9 h (time for first accumulation run). Also, the associated substrate consumption rates and PHA productivities were increased with the acclimation from 0.11 to 0.35 g COD/g Xaᵢ/h and from 44 to 87 mg PHA/g Xaᵢ/h after 4.9 h of accumulation time only, respectively.

The acclimation cycles led to an increase in feast response measured as the increase in RBCOD uptake rate (q) or as the reduction in specific feast duration (p) (FIG. 6). Already during the second feast-famine cycle, the RBCOD substrate uptake rate had increased 10% from that measured in the first cycle, i.e., q₂/q₁=1.11 and p₂/p₁=0.90. These results suggested that for this biomass, two and maximum three acclimation cycles would have sufficed as an acclimation step, thus increasing the overall substrate consumption and PHA production rates of the accumulation process with the benefits of increased biomass PHA content and PHA production.

In conclusion, we found that the PAP of a biomass harvested from a full-scale municipal wastewater treatment process, not a priori intended as a biomass source for PHA production, could be enhanced by applying an acclimation step. The acclimation step increased the feast response of the biomass to a distinct feedstock comprising a fermented-sludge-liquor RBCOD substrate used in the accumulation run, as quantified by an increase in qᵢ/q₁ or decrease in pᵢ/p₁ where i corresponds to the feast-famine cycle number.

### Example 6: Evaluation of Acclimation Effects on Stimulating Increased Substrate Uptake Rates on Activated Sludge Biomass Harvested from a Wastewater Treatment Process

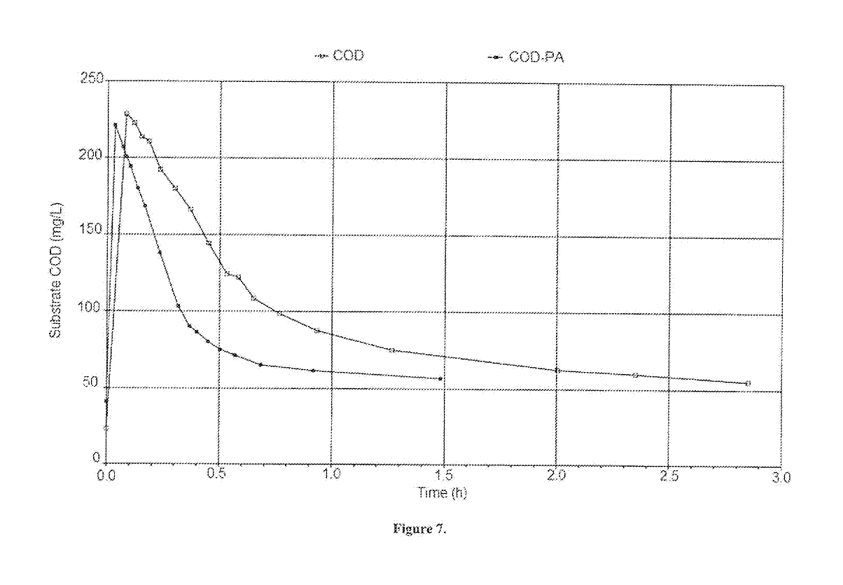

Two samples of activated sludge biomass from the same full-scale municipal wastewater treatment process described in Example 5 were subjected to batch feast tests at laboratory scale. One biomass sample was subjected to an acclimation step at pilot scale consisting of a pre-defined number of nine aerobic feast-famine cycles without biomass wasting and with an imposed feast-to-famine ratio of 0.125. The other biomass sample was not subjected to any acclimation step. The RBCOD for the acclimation step and batch feast tests consisted mostly of VFAs (98% of soluble COD) present in the liquor of fermented waste sludge (pH˜5; COD:N:P=100:10:0.045) used as substrate feedstock and produced at pilot scale as explained in Example 5. The acclimation step lasted a total of 13.8 h including 1.5 h of pre-aeration. During the batch feast tests, an aliquot of fermented-sludge liquor was added targeting a concentration of about 200 mg COD/L and the trend in COD removal was monitored (FIG. 7).

Similar as in Example 5, the acclimation feast-famine cycles increased the feast response of the biomass to the fermented-sludge-liquor RBCOD, and already during the second feast-famine cycle, the RBCOD substrate uptake rate (and feast length) increased (and decreased), respectively more than 30% from those measured in the first cycle, i.e., q₂/q₁=1.32 and p₂/p₁=0.76 (FIG. 8), respectively. A comparison of the biomass performance during the batch feast tests (FIG. 7) indicated that the biomass subjected to the acclimation step was able to take up the substrate COD at a higher rate and with higher associated oxygen uptake rates than the biomass without acclimation. Under the same substrate load, the biomass with acclimation removed around 70% of the substrate COD within 0.65 h, whereas the biomass without acclimation removed only 50% (FIG. 8).

In conclusion, an acclimation step applied to a biomass harvested from a full-scale municipal wastewater treatment process, not a priori intended for PHA-storage, induced an increase in biomass activity and substrate uptake rate of fermented-sludge-liquor RBCOD.

### Example 7: Evaluation of Trends of Biomass Respiration and Substrate Uptake Rates on Stimulating a Biomass Harvested from a Wastewater Treatment Process by Methods of Acclimation

Pilot-scale acclimation was applied to activated-sludge biomass that was harvested from a full-scale wastewater treatment process as described in Example 5. The pre-accumulation feedstock RBCOD was distinct from the wastewater treatment influent water quality and comprised acetic acid (7.540 gCOD/L) with NH₄Cl and KH₂PO₄ in amounts targeting a COD:N:P mass ratio of 100:1.4:0.13. The feed was pH adjusted to approximately 5 with the addition of NaOH. The acclimation process was applied to the harvested biomass after storage for 24 h including aerating the biomass in the mixed liquor for approximately 4 hours before applying the process of acclimation.

The biomass response, to repeatedly imposed cycles of feast-famine stimulation and with respect to trends in respiration and substrate uptake rates, was monitored. As for the other examples in this disclosure, (1) most of the biomass was retained during the acclimation process, (2) the duration of the overall acclimation time (in the order of hours) was significantly shorter than the solids retention time (SRT) used to produce the biomass (several days to a few weeks), and (3) the wastewater used to produce the biomass was distinct from the feedstock to be used for PHA production following an intermediary step of acclimation. The timing of the repeated feast-famine stimulation was driven by the biomass response wherein the feast time to remove a pulse addition of feedstock to reach 100 mg-COD/L was estimated based on the trend of increase and decrease of biomass respiration rate. The respective acclimation aerobic famine times were made to be 8 times their respective feast times. Thus, a feast-to-famine ratio of 0.125 was applied. The acclimation feast-famine cycles were operated similarly as described in Example 1, in terms of feast conditions with respect to peak RBCOD concentration. The analytical techniques used were the same as specified in Example 1.

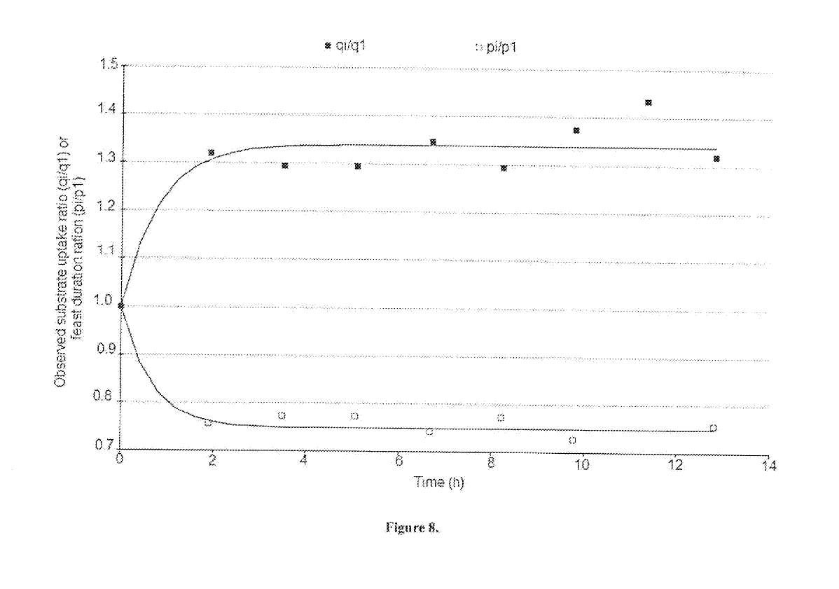

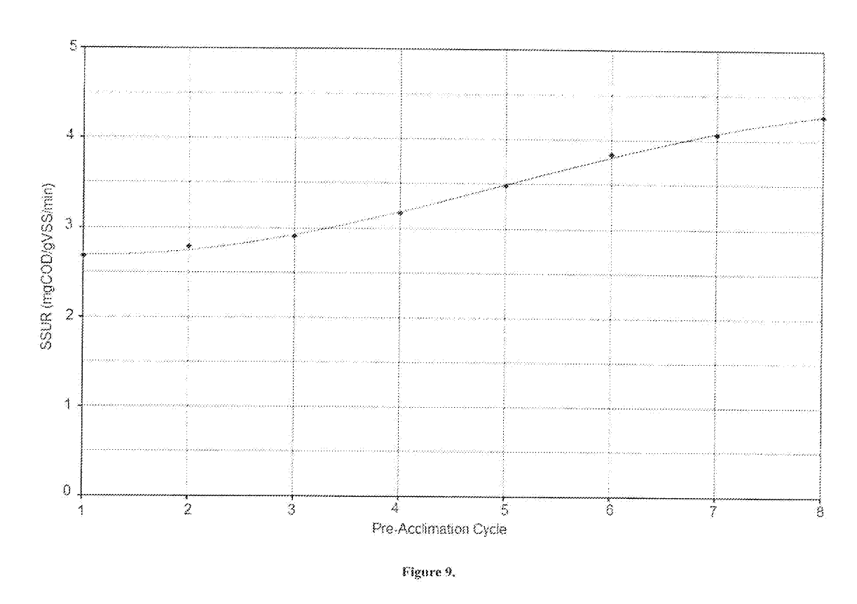

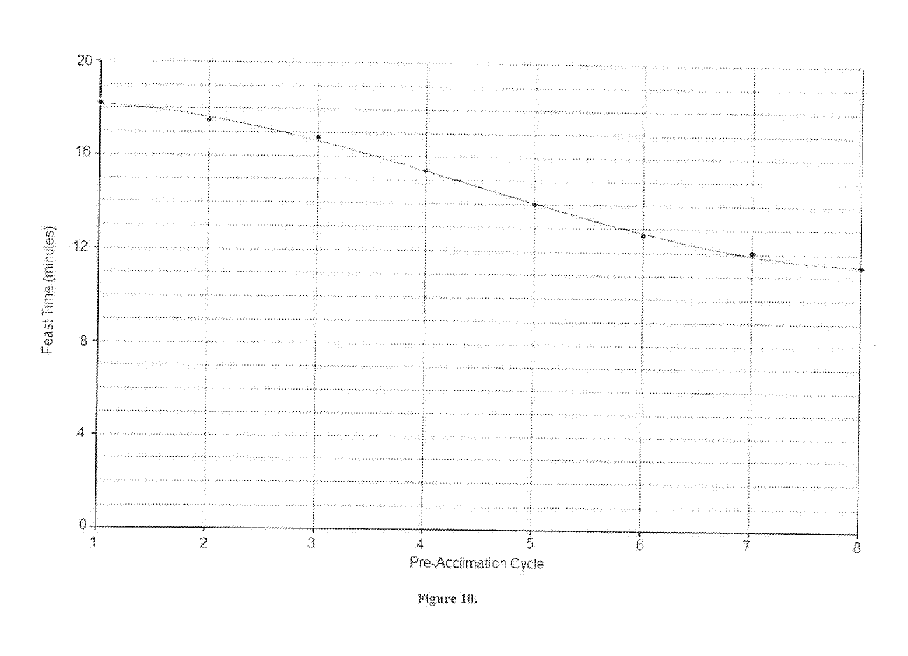

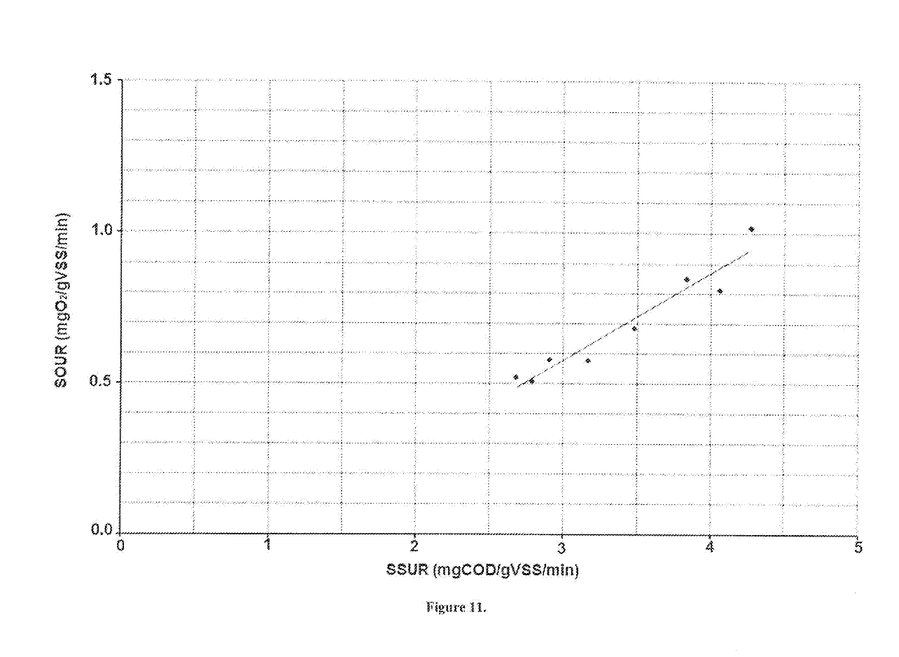

With the first feast stimulation, the biomass was with an estimated specific oxygen uptake rate (SOUR₁) of 0.52 mgO₂/gVSS/min and a specific substrate uptake rate (SSUR₁) of 2.68 mgCOD/gVSS/min. The initial feast (t_{fe}) time was 18.2 minutes. The feast time is directly related to the substrate uptake rate, so that the relative change in biomass response to repeated cycles of acclimation feast stimulations may be monitored similarly with respect to the parameter ratio (Pᵢ/P₁) using the feast time, or (qᵢ/q₁) using the parameter of SSUR. Over the 8 applied feast stimulations, the biomass response can be seen to approach limiting values of increase in SSUR (FIG. 9) or decrease in t_{fe} (FIG. 10). With reference to FIGS. 9 and 10, qᵢ/q₁ increased from 1.0 to 1.6 and Pᵢ/P₁ decreased from 1.0 to 0.6 over the 8 acclimation feast-famine cycles. Successive stimulation increases in biomass SSUR were proportional to increases in the biomass SOUR (FIG. 11), suggesting that respiration rate and substrate uptake rate may similarly be used as indicators of an effect of acclimation.

The present invention may, of course, be carried out in other ways than those specifically set forth herein without departing from essential characteristics of the invention. The present embodiments are to be considered in all respects as illustrative and not restrictive, and all changes coming within the meaning and equivalency range of the appended claims are intended to be embraced therein.

1-36. (canceled)

37. A method of treating wastewater and producing polyhydroxyalkanoates (“PHA”), comprising:
biologically treating a first wastewater stream having a readily biodegradable chemical oxygen demand (RBCOD) content and producing a treated effluent and biomass;
providing a second wastewater stream having RBCOD content, the second wastewater stream having a different RBCOD content as compared to the first wastewater stream;
using the biomass produced by the first wastewater stream to produce PHA with the second wastewater stream and biologically treat the second wastewater stream;
before producing PHA with the second wastewater stream, acclimating the biomass to the second wastewater stream by:
stimulating the biomass into a period of feast respiration by subjecting the biomass to a fraction of wastewater from the second wastewater stream;
during the period of feast respiration, measuring directly or indirectly the substrate uptake rate or biomass respiration rate;
after the biomass removes at least 90% of the RBCOD from said faction of wastewater, subjecting the biomass to a period of famine for a period of time at least twice as long as the preceding period of feast respiration;
repeatedly subjecting the biomass to periods of feast respiration and periods of famine for at least two feast respiration-famine cycles and until either the RBCOD uptake rate is at least 10% higher than the RBCOD uptake rate during a first period of feast respiration, or the respiration rate is at least 10% higher than the respiration rate during the first period of feast respiration; and
thereafter biologically treating the second wastewater stream while accumulating and thereby producing PHA in a batch process by subjecting the biomass to a prolonged feast respiration with the second wastewater stream.

38. The method of claim 37, further comprising: measuring the biomass respiration rate during periods of famine; and maintaining the biomass respiration rate during periods of famine such that the respiration rate is less than 50% of the extant maximum potential biomass respiration rate.

39. The method of claim 37, further comprising:
measuring the solids retention time (SRT) for the biomass while biologically treating the first wastewater stream;
limiting the time for acclimating the biomass to the second wastewater stream, such that the time for acclimating the biomass to the second wastewater stream is less than the SRT for biologically treating the first wastewater stream.

40. The method of claim 37, wherein stimulating the biomass into a period of feast respiration by subjecting the biomass to a fraction of wastewater from the second wastewater stream occurs in a stimulating zone and wherein subjecting the biomass to a period of famine for a period at least twice as long as the period of feast occurs in a maintenance zone.

41. The method of claim 37 including limiting biomass growth and biomass removal in the acclimation process by:
i. retaining most of the biomass in the process during acclimation; and
ii. wherein the amount of biomass in the acclimation process does not increase by more than a fraction of the amount of biomass added to the acclimation process.

42. The method of claim 37 wherein during acclimating the biomass to the second wastewater stream, limiting the growth of the biomass such that the amount of biomass in the acclimation process does not increase by more than a fraction of the amount of biomass added to the process.

43. A process for producing PHA comprising:
obtaining biomass produced in the course of biologically treating a first wastewater source containing a readily biodegradable chemical oxygen demand (RBCOD);
subjecting the biomass to a PHA accumulation process in a second wastewater source having a different RBCOD content from that of the first wastewater source;
prior to subjecting the biomass to the PHA accumulation process, enhancing the PHA accumulation potential of the biomass by subjecting the biomass to an acclimation process in the second wastewater source by:
i. subjecting the biomass to repeated feast-famine periods by exposing the biomass during each feast period to a fraction of the second wastewater source;
ii. measuring directly or indirectly the RBCOD uptake by the biomass or biomass respiration rate during the feast period;
iii. after the biomass has removed 90% or more of the RBCOD in the fraction of the second wastewater source, subjecting the biomass to a period of famine;
iv. maintaining the famine period for a length of time that is at least two times greater than the length of time of the proceeding feast period; and
after at least two feast-famine cycles in the acclimation process and after one or more measured parameters reveal an increased RBCOD uptake rate of the biomass during a feast period, or an increased biomass respiration rate during a feast period, subjecting the biomass to a PHA accumulation process by utilizing the second wastewater source to produce and accumulate PHA in the biomass derived from treating the first wastewater source.

44. The method of claim 43, further comprising limiting PHA accumulation during the acclimation process such that no more than 15% of the biomass weight is accumulated PHA.

45. The method of claim 43, wherein the fraction of the second wastewater stream stimulates a feast respiration response by exposing the biomass or fractions of the biomass at any given time to a peak RBCOD concentration that is between 10 mg-COD/L and 2,000 mg-COD/L.

46. The method of claim 43 including limiting biomass growth and biomass removal in the acclimation process by:
i. retaining most of the biomass in the process during acclimation; and
ii. wherein the amount of biomass in the acclimation process does not increase by more than a fraction of the amount of biomass added to the acclimation process.

47. The method of claim 43 wherein the biomass response in the acclimation process is measured directly or indirectly as a parameter related to the rate of respiration or the rate of RBCOD uptake (q), and the number of feast stimulations in the acclimation process is such that there is an increase in the rate (q) by at least 10% with respect to the rate measured in a first feast stimulation (q1).

48. A method of biologically treating two different wastewater streams and producing PHA in the process, comprising:
providing a first wastewater stream and a second wastewater stream that are different in readily biodegradable chemical oxygen demand (RBCOD) content;
directing the first wastewater stream into a biological treatment system and biologically treating the first wastewater stream and producing biomass and a first treated effluent;
separating at least some of the biomass from the first wastewater stream;
enhancing the PHA accumulation potential of the separated biomass by subjecting the separated biomass to an acclimation process using the second wastewater stream, the acclimation process carried out generally parallel to the biological treatment of the first wastewater stream and including:
i. subjecting the biomass to repeated feast-famine periods by exposing the biomass during each feast period to a fraction of the second wastewater stream;
ii. measuring directly or indirectly the RBCOD uptake by the biomass and/or biomass respiration rate during the feast period;
iii. after the biomass has removed 90% or more of the RBCOD in the fraction of the second wastewater stream, subjecting the biomass to a period of famine;
iv. maintaining the famine period for a length of time that is at least two times greater than the length of time of the proceeding feast period; and
during the acclimation process, reducing the RBCOD concentration in the second wastewater stream to produce a second treated effluent and directing the second treated effluent from the acclimation process; and
after at least two feast-famine cycles in the acclimation process and after one or more measured parameters reveal an increased RBCOD uptake rate of the biomass during a feast period, or an increased biomass respiration rate during a feast period, subjecting the biomass to a PHA accumulation process by utilizing the second wastewater stream to produce and accumulate PHA in the biomass which was derived previously from the first wastewater stream.

49. The method of claim 48 including splitting the second wastewater stream into third and fourth wastewater streams and directing the third wastewater stream into the acclimation process and directing the fourth stream to the PHA accumulation process.
